(19) United States
(12) Patent Application Publication
(10) Pub. No.: US 2007/0082870 A1
(43) Pub. Date: Apr. 12, 2007

(54) Pharmaceutical formulations of cyclodextrins and antifungal azole compounds

(76) Inventors: Charles Michael Buchanan, Kingsport, TN (US); Norma Lindsey Buchanan, Kingsport, TN (US); Juanelle Little Lambert, Gray, TN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/545,516

(22) Filed: Oct. 11, 2006

Related U.S. Application Data
(60) Provisional application No. 60/724,792, filed on Oct. 11, 2005.

(51) Int. Cl. A61K 31/724; A61K 31/496

(52) U.S. Cl. 514/58; 514/254.07

(57) ABSTRACT

This invention relates to methods of increasing the aqueous solubility of an antifungal azole using hydroxybutenyl cyclodextrins. This invention also relates to method of increasing the bioavailability of an antifungal azole compounds administered to subjects.

This application claims the benefit of priority of U.S. Provisional Patent Application Ser. No. 60/724,792, filed Oct. 11, 2005.

### FIELD OF THE INVENTION

This invention relates to methods of increasing the aqueous solubility of an antifungal azole using hydroxybutenyl cyclodextrins. This invention also relates to method of increasing the bioavailability of an antifungal azole compounds administered to subjects.

### BACKGROUND OF THE INVENTION

Cyclodextrins (CDs) are cyclic oligomers of glucose joined by α-1,4 linkages. The most common cyclodextrins contain 6, 7, or 8 glucose monomers and are commonly called α-CD, β-CD, and γ-CD, respectively. Higher oligomers containing up to 12 glucose monomers are also known. Topologically, CD can be represented as a toroid in which the primary hydroxyls are located on the smaller circumference, and the secondary hydroxyls are located on the larger circumference. Because of this arrangement, the interior of the torus is hydrophobic while the exterior is sufficiently hydrophilic to allow the CD to be dissolved in water. This difference between the interior and exterior faces allows the CD to act as a host molecule and to form inclusion complexes with guest molecules, provided the guest molecule is of the proper size to fit in the cavity. The CD inclusion complex can then be dissolved in water thereby providing for the introduction of guest molecules that have little or no aqueous solubility into an aqueous environment. Reviews of CD complexes can be found in Chem. Rev., 1997, 97,1325-1357 and in Supramolecular Chemistry, 1995, 6, 217-223.

Unmodified cyclodextrins, especially β-cyclodextrin, have limited aqueous solubility, and can crystallize from solution, particularly after renal concentration. This means that the ability of unmodified cyclodextrins to solubilize and stabilize guest molecules in an aqueous environment is limited. Furthermore, unmodified cyclodextrins, e.g. β-cyclodextrin, have been shown to cause renal and liver damage after parenteral administration. These issues have led to exploration of the use of chemically modified or derivatized cyclodextrins that avoid these problems. Two examples of derivatized cyclodextrins are hydroxybutenyl cyclodextrins (HBenCD), which are disclosed in U.S. Pat. No. 6,479,467 (2002) and in Carbohydrate Research, 2002, 327(6), 493-507, and sulfonated hydroxybutenyl cyclodextrins (SulfoHBenCD), which are disclosed in U.S. Pat. No. 6,479,467. These CD derivatives solve many of the issues noted for the parent unmodified CDs.

Unmodified cyclodextrins and cyclodextrin derivatives have been used to increase the solubility, dissolution rate, and/or stability of many different types of compounds. It has also been found that for many compounds, complexation with cyclodextrins either is not possible or yields no advantage. Structurally different cyclodextrin derivatives can also provide dramatically different solubilization rates, dissolution rates, and stabilization of drugs under otherwise similar conditions (cf., Cyclodextrins in Drug Formulations: Part II, Pharmaceutical Technology, 1991, 24-38).

One treatment of cutaneous and systemic fungal infections has been by the administration of antifungal azole compounds. Antifungal azole compounds are structurally diverse and are characterized by having imidazole or a triazole functionalities. This class includes such drugs as itraconazole, ketoconazole, fluconazole, saperconazole, miconazole, ravunconazole, posaconazole, voriconazole and several others. Many compounds in this class, although highly effective antifungal agents, are not widely used clinically because of their poor water solubility and, as a result, poor bioavailability.

### SUMMARY OF THE INVENTION

Applicants have developed methods of increasing the aqueous solubility of an antifungal azole compound comprising forming a complex or mixture of at least one antifungal azole compound with a hydroxybutenyl cyclodextrin.

In certain embodiments, the hydroxybutenyl cyclodextrin is hydroxybutenyl-α, β, or γ-cyclodextrin. In other embodiments, the hydroxybutenyl cyclodextrin is hydroxybutenyl-β-cyclodextrin.

In some embodiments, the hydroxybutenyl cyclodextrin has a molar substitution of about 1 to about 12, or from about 3 to about 10, or from about 4 to about 7. In other embodiments, the hydroxybutenyl cyclodextrin Is water soluble.

In certain embodiments, the hydroxybutenyl cyclodextrin derivative is sulfonated hydroxybutenyl cyclodextrin; in others, sulfonated hydroxybutenyl-α, β, or γ-cyclodextrin hydroxybutenyl; and in others, sulfonated hydroxybutenyl-β-cyclodextrin.

In some embodiments, the sulfonated hydroxybutenyl cyclodextrin has a molar substitution-of sulfonate of about 0.02 to about 12, and in other embodiments, from about 0.1 to about 2.

In certain embodiments, the antifungal azole compound is an imidazole or triazole compound. In other embodiments, the antifungal azole compound is albaconazole, becliconazole, bifonazole, bilastine, butoconazole, carbendazim, clotrimazole, doconazole, eberconazole, econazole, fenticonazole, fluconazole, flutrimazole, fosfluconazole, genaconazole, imiquimod, itraconazole, ketoconazole, lanoconazole, liarozole, luliconazole, metronidazole, miconazole, mycoprex, omoconazole, parconazole, posaconazole, ravunconazole, resiquimod, rifaximin, samidazole, saperconazole, seraconazole, terconazole, or voriconazole or structural analogs or metabolites thereof. In alternative embodiments, the antifungal azole compound is itraconazole, voriconazole, ravunconazole, or posaconazole.

In alternative embodiments, the aqueous solubility of the antifungal azole compound as a complex or mixture with the hydroxybutenyl cyclodextrin is from about 1.1 to about 1000 times greater, from about 2 to about 100 times greater, from about 2 to about 50 times greater, from about 2 to about 25 times greater, or from about 2 to about 5 times greater than the aqueous solubility of the antifungal azole compound without the hydroxybutenyl cyclodextrin.

Another aspect of the invention is a method of increasing the aqueous solubility of an antifungal azole compound comprising forming a complex or mixture of an antifungal azole compound with a hydroxybutenyl cyclodextrin in the presence of a chiral compound or salt. In certain embodiments, the method further comprises matching the stereochemical configuration of the antifungal azole compound, the hydroxybutenyl cyclodextrin and the chiral compound.

In alternative embodiments of the invention, the chiral compound is citric acid or a salt thereof, tartaric acid or a salt thereof, D-tartaric acid or a salt thereof, or L-tartaric acid or a salt thereof.

Another aspect of the invention is a method of increasing the bioavailability of an antifungal azole compound comprising administering a complex or mixture of an antifungal azole compound with a hydroxybutenyl cyclodextrin produced by the methods of the invention to a subject. In certain embodiments, the subject is a human.

In certain embodiments of the invention, the complex or mixture of an antifungal azole compound with a hydroxybutenyl cyclodextrin administered to a subject further comprises an amine-containing polymer. In alternative embodiments, the amount of amine-containing polymer present in the complex or mixture is from about 3 wt % to about 40 wt % or from about 5 wt % to about 30 wt %. In some embodiments, the amine-containing polymer is chitosan or a derivative thereof.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed. The accompanying drawings, which are incorporated in and constitute part of this specification, and together with the description, serve to explain the principles of the invention.

### BRIEF DESCRIPTION OF THE DRAWING

FIG. 1 shows the equilibrium solubility of itraconazole in water and buffers at 10 wt % cyclodextrin concentration. Abbreviations: HBenBCD, hydroxybutenyl-β-cyclodextrin; HPBCD, hydroxypropyl-β-cyclodextrin; SBEBCD, sulfobutyl-β-cyclodextrin.

FIG. 2 shows the equilibrium solubility of itraconazole in water and pH 3 phosphate buffers from 0 to ca. 25 wt % cyclodextrin concentrations. Abbreviations: HBenBCD, hydroxybutenyl-β-cyclodextrin; HPBCD, hydroxypropyl-β-cyclodextrin; SBEBCD, sulfobutyl-β-cyclodextrin.

FIG. 3 shows the equilibrium solubility of ketoconazole in water and buffers at 10 wt % hydroxybutenyl-β-cyclodextrin concentration (HBenBCD).

FIG. 4 shows the equilibrium solubility of voriconazole in water and buffers at 10 wt % hydroxybutenyl-β-cyclodextrin concentration (HBenBCD).

FIG. 5 shows the equilibrium solubility of voriconazole in pH 3 succinate buffer from 0 to ca. 40 wt % cyclodextrin concentrations. Abbreviations: HBenBCD, hydroxybutenyl-β-cyclodextrin; SBEBCD, sulfobutyl-β-cyclodextrin.

FIG. 6 shows equilibrium solubility of clotrimazole in water from 0 to ca. 25 wt % cyclodextrin concentrations. Abbreviations: HBenBCD, hydroxybutenyl-β-cyclodextrin; HPBCD, hydroxypropyl-β-cyclodextrin.

FIG. 7 shows the dissolution profiles of itraconazole:HBenBCD complexes.

FIG. 8 shows a time versus concentration plot from a study involving IV and oral administration of itraconazole:HBenBCD complexes to Sprague-Dawley rats.

### DETAILED DESCRIPTION OF THE INVENTION

The present invention may be understood more readily by reference to the following detailed description of the invention and the examples provided therein. It is to be understood that this invention is not limited to the specific methods, formulations, and conditions described, as such may vary. It is also to be understood that the terminology used herein is for the purpose of describing particular aspects of the invention only and is not intended to be limiting.

A. Definitions

In order that the present invention may be more readily understood, certain terms are first defined. Additional definitions are set forth throughout the detailed description.

The singular forms “a,” “an,” and “the” include plural referents unless the context clearly dictates otherwise.

The term “hydroxybutenyl cyclodextrin” refers to all forms of hydroxybutenyl cyclodextrins, including hydroxybutenyl-α, β, or γ-cyclodextrins as well as higher oligomers containing up to about twelve glucose monomers. The term “hydroxybutenyl cyclodextrin” also encompasses derivatives thereof, including sulfonated hydroxybutenyl cyclodextrins such as, for example, sulfonated hydroxybutenyl-α, β, or γ-cyclodextrins.

The term “hydroxybutenyl cyclodextrin derivatives” refers to hydroxybutenyl cyclodextrins that have been further elaborated by attachment of substituents to the hydroxyls of the cyclodextrin ring and/or hydroxybutenyl substituent or by manipulation of the olefin of the hydroxybutenyl substituent.

The term “metabolites” refers to compounds (e.g., active species) produced upon introduction of the compounds of the invention into a biological system.

The term “analogs” refers to structurally similar compounds that share at least one biological property.

B. Methods

In one aspect, the present invention relates to methods of increasing the aqueous solubility of an antifungal azole compound by forming a complex or mixture of an antifungal azole compound with a hydroxybutenyl cyclodextrin.

In one embodiment, the aqueous solubility of an antifungal azole compound as a complex or mixture with a hydroxybutenyl cyclodextrin can be from about 1.1 to about 1000 times greater than the aqueous solubility of the antifungal azole compound without the hydroxybutenyl cyclodextrin. In another embodiment, from about 2 to about 100 times greater. In additional embodiments, from about 2 to about 50 times greater, from about 2 to about 25 times greater, or from about 2 to about 5 times greater.

The compositions of the present invention include hydroxybutenyl cyclodextrins or derivatives thereof and at least one antifungal azole compound. In some embodiments, the hydroxybutenyl cyclodextrins can be hydroxybutenyl-α, β, or γ-cyclodextrins. In other embodiments, the hydroxybutenyl-β-cyclodextrins can have a molar substitution (MS, wherein MS is the total number of substituents attached to the CD) from about 1 to about 12. In additional embodiments, the hydroxybutenyl-β-cyclodextrins can have a MS from about 3 to about 10. In further embodiments, the hydroxybutenyl-β-cyclodextrins can be water-soluble and can have a MS from about 4 to about 7.

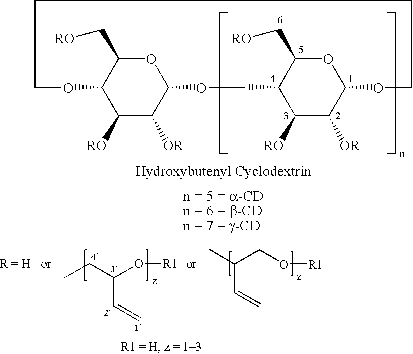

In certain embodiments, the hydroxybutenyl cyclodextrin derivatives can be sulfonated hydroxybutenyl-α, β, or γ-cyclodextrins. In some embodiments, the sulfonated hydroxybutenyl cyclodextrins can be sulfonated hydroxybutenyl-β-cyclodextrins comprising at least one hydroxybutyl sulfonate substituent. In other embodiments, the sulfonated hydroxybutenyl-β-cyclodextrins can have a MS of hydroxybutyl sulfonate from about 0.02 to about 7 or the hydroxybutenyl-β-cyclodextrins can have a MS of hydroxybutyl sulfonate from about 0.1 to about 2. In the case of sulfonated hydroxybutenyl-α, β, or γ-cyclodextrins, those skilled in the art will recognize that these cyclodextrin ethers may contain both hydroxybutenyl substituents and hydroxybutyl sulfonate substituents. In this case, the total MS is provided by the sum of the hydroxybutenyl MS and the hydroxybutyl sulfonate. The typical total MS is from about 0.02 to about 12. In some embodiments, the cyclodextrin ethers containing at least one hydroxybutyl sulfonate substituent may also further comprise additional alkyl, sulfinate, or disulfonate substituents.

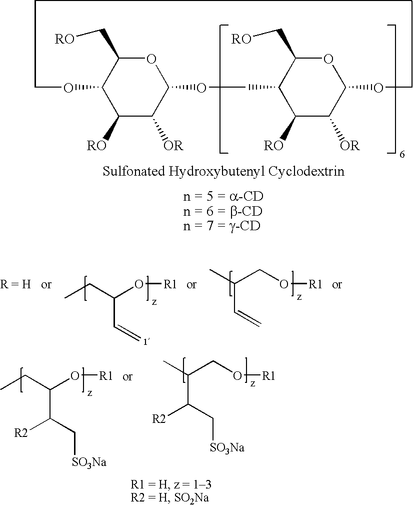

The compositions of the present inventions also include one or more antifungal azole compounds, structural analogs or a pharmaceutically acceptable salt or metabolites thereof. Antifungal azole compounds are structurally diverse and are characterized by having imidazole or a triazole functionalities. In some embodiments, the antifungal azole compound can be albaconazole, becliconazole, bifonazole, bilastine, butoconazole, carbendazim, clotrimazole, doconazole, eberconazole, econazole, fenticonazole, fluconazole, flutrimazole, fosfluconazole, genaconazole, imiquimod, itraconazole, ketoconazole, lanoconazole, liarozole, luliconazole, metronidazole, miconazole, mycoprex, omoconazole, parconazole, posaconazole, ravunconazole, resiquimod, rifaximin, samidazole, saperconazole, seraconazole, terconazole or voriconazole or structural analogs or metabolites thereof. In other embodiments, the antifungal azole can be itraconazole, ketoconazole, clotrimazole, fluconazole, saperconazole, miconazole, ravunconazole, voriconazole or posaconazole. In additional embodiments, the antifungal azole can be itraconazole, ravunconazole, voriconazole or posaconazole.

The amount of antifungal azole compound or a pharmaceutically acceptable salt or metabolite thereof used is typically an amount sufficient such that the formulation provides the desired therapeutic effect. In some embodiments, they can be administered one to four times a day with a unit dosage of 0.25 to 500 milligrams (mg) in human patients. This dosage can be varied depending on the age, body weight and medical condition of the patient and the type of administration. One dose of 50-200 mg one time a day can be used in some embodiments.

The complexes and mixtures of the present invention may increase the bioavailability of an antifungal azole compound, allowing for smaller doses to achieve therapeutic responses comparable to larger doses of the antifungal azole compound alone. The amount of antifungal azole compound in a complex or mixture with a hydroxybutenyl cyclodextrin administered to a subject may need to be modified accordingly to account for changes in bioavailability.

The compositions of the present invention may be in any physical phase, including solid, liquid, and semisolid. Examples of solid compositions include, but are not limited to, tablets, capsules, or oral powders. In some embodiments, a dry, solid physical mixture of HBenCD or SulfoHBenCD and an antifungal azole compound or a dry, solid inclusion complex of HBenCD or SulfoHBenCD and an antifungal azole compound can be used, for example, to fill a capsule or compressed into a tablet for administration. Dry, solid inclusion complexes can be used in some embodiments. Upon exposure to an aqueous environment of use, such as the luminal fluid of the gastrointestinal tract or the salivary fluid of the buccal cavity, the solubility and hence bioavailability of the drug is increased relative to the drug in the absence of HBenCD and SulfoHBenCD.

Liquid formulations include aqueous solutions, and solutions in water soluble organic compounds, or combinations thereof. In the case of water soluble organic compounds, any type of formulation may be used as disclosed in the commonly assigned U.S. Provisional Patent Application 60/626,005, entitled “Cyclodextrin Solubilizers for Liquid and Semi-Solid Pharmaceutical Formulations”, which is incorporated by reference herein in its entirety. In some embodiments, aqueous solutions may be those in which the water content is at least 20 wt %.

On a weight basis in solid formulations, the ratio of antifungal azole compound to HBenCD or SulfoHBenCD can be, for example, from about 1:120 to about 3:1. In some embodiments, the ratio can be from about 1:40 to about 2:1. In others, the molar ratio can be from about 1:20 to about 1:1 w/w.

In some embodiments, aqueous solutions comprising HBenCD or SulfoHBenCD, an antifungal azole compound, and sterile water or other pharmaceutically acceptable aqueous medium may be sufficient to form product solutions which can be directly administered, for example parenterally or subcutaneously, directly to human patients. Due the stability provided by HBenCD or SulfoHBenCD, solutions in some embodiments can be stored under appropriate conditions (from about 5° C. to about room temperature) for periods up to 2 years or longer. In other embodiments, an isolated complex can be stored under appropriate conditions at room temperature for periods up to 2 years are longer, and reconstituted into a product solution as needed. The product solution can be prepared by dissolving the solid inclusion complex in water or other pharmaceutically acceptable aqueous medium in an amount sufficient to generate a solution of the required strength for oral or parenteral administration.

The compositions of the present invention optionally include additional components. In some embodiments, additional components may be useful in achieving or enhancing desired properties of the compositions. Examples of such components include, but are not limited to, surfactants, fillers, disintegrants, binders, lubricants, dispersing agents, surfactants, thickening agents, as well as other excipients such as cellulose esters and ethers, starch or starch derivatives, chitosan or chitosan derivatives, dyes, and flavorings.

Liquid formulations optionally contain buffers, antioxidants, preservatives and tonicity adjusters. Examples of buffers include, but are not limited to, phosphates, acetates, citrates, benzoates, succinates, bicarbonates, and glycine. Examples of antioxidants include ascorbic acid, sodium bisulfite, sodium metabisulfite, monothioglycerol, thiourea, butylated hydroxytoluene, butylated hydroxy anisole, and ethylenediaminetetraacetic acid salts. Preservatives useful in liquid formulations include benzoic acid and its salts, sorbic acid and its salts, alkyl esters of parahydroxybenzoic acid, phenol, chlorobutanol, benzyl alcohol, thimerosal, benzalkonium chloride and cetylpyridinium chloride. The buffers mentioned previously as well as dextrose, glycerin, potassium chloride, and sodium chloride can be used for tonicity adjustment if necessary.

In one embodiment, the additional formulation components are amine-containing polymers with a pKa different from that of the azole antifungal compound. The amine containing polymers can act as a proton reservoir in these formulations and serve to stabilize the basic drug, slowing the rate of drug precipitation or crystallization. Such an effect can serve as a means to increase the drug absorption window and increase oral bioavailability. In one embodiment, the amine-containing polymer can be chitosan or derivatives thereof.

The amount of amine-containing polymer in the composition is typically an amount sufficient to increase the drug absorption window and increase oral bioavailability of the antifungal azole compound. In some embodiments, the amount of amine-containing polymer in the composition is from about 3 wt % to about 40 wt %. In other embodiments, the amount of amine-containing polymer in the composition is from about 5 wt % to about 30 wt %.

Additional formulation components may include surfactants. Suitable surfactants include water soluble or dispersible non-ionic surfactants. Other suitable surfactants include cremophors (eg. Cremophor EL, Cremophor 40), polysorbate 20 (Tween 20), polysorbate 80 (Tween 80), tocopherol polyethylene glycol 1000 succinate (TPGS), solutol HS-15, sorbitan monooleate. (Span 80), alkylpolyglycosides (cf. U.S. Pat. No. 6,077,945), and carbohydrate esters (e.g. sucrose acetate isobutyrate). Often, the surfactants are selected from Tween 20, Tween 80, TPGS, sucrose acetate isobutyrate or alkylpolyglycosides.

In the present invention, surfactants can serve to modify the stability and release rates of the drugs in the physiological environment. In certain instances, the surfactant can also act as a P-glycoprotein inhibitor. A suitable concentration of surfactant in the pharmaceutical formulations of the present invention can range from about −1 5X to about +3X of the critical micelle concentration of the surfactant. Another concentration of surfactant can range from about −10X to about +1X of the critical micelle concentration of the surfactant.

The pharmaceutical formulations may optionally comprise β-glycoprotein (p-gp) inhibitors. In some instances, the water-soluble organic solvent, the cyclodextrin derivative, one or more of the drug components, or the surfactant can also serve the dual purpose of a p-gp inhibitor. One example is methyl cyclodextrin, which is known to be a p-gp inhibitor. In another example, polyethylene glycol-polyethylene glycol block copolymers (eg. Pluronics available from BASF), cremophors, TPGS, Solutol, and Polysorbate 80 are known p-gp inhibitors.

Formulation pH, buffering capacity, and ionic strength are all considered in preparing compositions of the present invention. Antifungal azole compounds typically are weak bases that can be ionized in appropriate media. The pH of the formulation media can be adjusted by any effective agent. Several such agents are known to those skilled in the art, including but not limited to, organic acids, organic bases, or buffers. Examples of organic acids include but are not limited to formic, acetic, propionic, trifluoroacetic, citric, maleic, tartaric, ascorbic, methanesulfonic, benzenesulfonic, toluenesulfonic acids. Examples of organic bases include, but are not limited to, ethylene diamine, triethanolamine, tris(hydroxymethyl)aminomethane, and butyl amine. Some examples of buffers include phosphates, acetates, citrates, tartrates, benzoates, succinates, bicarbonates, and glycine. In some embodiments, the concentration of organic acids can be from about 0.5 N to about 0.001 N. In others, the concentration of organic acids can be from about 0.2 N to about 0.01 N or from about 0.1 N to about 0.05 N. In the case of buffers, the normality in some embodiments can be from about 0.5 N to about 0.001 N or from about 0.1 N to about 0.01 N or even from about 0.05 N to about 0.02 N. With regard to ionic strength, in some embodiments the ionic strength can be less than about 200 mM. In other embodiments, the ionic strength can be less than about 100 mM or sometimes less than about 100 mM. In some embodiments, increases in ionic strength may reduce solubility.

In another aspect, the invention provides a method for increasing the aqueous solubility of an antifungal azole compound comprising forming mixtures or complexes of an antifungal azole compound with hydroxybutenyl cyclodextrin or derivative thereof in the presence of at least one chiral compound or salt. The stereochemistry of the chiral compound or salt may be matched with that of the hydroxybutenyl cyclodextrin or derivative thereof and that of the drug, thereby increasing drug solubility beyond that normally expected. In one embodiment, the drug can be a nonracemic compound. In another, the chiral compound or salt can be citric acid or tartaric acid or salts thereof, or the chiral compound or salt can be L-tartaric acid, D-tartaric acid or salts thereof.

The invention further includes methods of making the compositions of the present invention. Liquid formulations of HBenCD or SulfoHBenCD and antifungal azole compounds, in some embodiments, can be formed by conventional methods. See U.S. Pat. Nos. 6,479,467 and 6,479,467. For example, the inclusion complex can be formed in situ by adding an antifungal azole compound, in an amount less than or equal to the amount corresponding to equilibrium solubility, directly to a solution of HBenCD or SulfoHBenCD in water or other pharmaceutically acceptable aqueous medium. Alternatively, the antifungal azole compound can be dissolved in appropriate water-soluble organic compounds and mixed with the CD solution. In some embodiments, a dry, solid inclusion complex of antifungal azole compounds with hydroxybutenyl cyclodextrin or derivative thereof can be formed by the methods of the present invention. Or an excess amount of an antifungal azole compound can be added to an aqueous solution of HBenCD or SulfoHBenCD and mixed for a period of time sufficient to obtain equilibrium solubility. Excess drug is removed, and the inclusion complex isolated by drying techniques such as spray drying or freeze drying. In some embodiments, the inclusion complex can be isolated by precipitation in a solvent in which the complex has minimal solubility.

In one embodiment, a dry, solid inclusion complex of an antifungal azole compound with hydroxybutenyl cyclodextrin or derivative thereof can be formed by preparing multiple solutions. For example, a solution of the CD derivative in aqueous media and a solution of the antifungal azole compound in a water-soluble organic liquid. Mixing of the two solutions, adjustment of solution pH by appropriate methods, followed by isolation by drying techniques provides the solid complex.

In some embodiments, dry, solid physical mixture of HBenCD or SulfoHBenCD and an antifungal azole compound can be formed by any effective method. Examples of such methods include, but are not limited to, those that provide an intimate physical mixture in which the particle size of the components is reduced. For example, methods such as dry milling can be utilized in the present invention. The molar ratio of the inclusion complex components can vary depending upon the initial solution concentration of each component. In some embodiments, the amount of HBenCD or SulfoHBenCD is such that the molar ratio of antifungal azole compound to cyclodextrin derivative can be from about 1:0.1 to about 1:30. In other embodiments, the molar ratio can be from about 1:0.5 to about 1:10, or the molar ratio can be from about 1:1 to about 1:4.

The present invention also includes methods of administration of the compositions of the present invention to mammals, including but not limited to humans. Any method of administration can be used. Examples of such methods include, but are not limited to, oral administration (e.g. buccal or sublingual administration), oral ingestion, anal administration, aerosol administration, intraperitoneal administration, intravenous administration, transdermal administration, intradermal administration, subdermal administration, intramuscular administration, intrauterine administration, vaginal administration, administration into a body cavity, surgical administration at the location of a tumor or internal injury, administration into the lumen or parenchyma of an organ, and parenteral administration.

Any technique can be used in the method of administration. Examples of techniques useful in the various forms of administration above include, but are not limited to, topical application, ingestion, surgical administration, injections, sprays, transdermal delivery devices, osmotic pumps, depositing directly at a desired site, or other means familiar to one of ordinary skill in the art. Sites of application can be external, such as on the epidermis, or internal, for example a gastric ulcer, a surgical field, or elsewhere.

The compositions of the present invention can be applied in any form. Examples include, but are not limited to, creams, gels, solutions, suspensions, liposomes, particles, or other means known to one of skill in the art of formulation and delivery of therapeutic and cosmetic compounds. Appropriate formulations for subcutaneous administration can be implants, depot, needles, capsules, and osmotic pumps. Examples of appropriate formulations for vaginal administration include, but are not limited to, creams and rings. Some examples of appropriate formulations for oral administration include, but are not limited to, tablets, capsules, liquids, syrups, and suspensions. Some examples of appropriate formulations for transdermal administration include, but are not limited to, gels, creams, pastes, patches, sprays, and gels. Examples of appropriate delivery mechanisms for subcutaneous administration include, but are not limited to, implants, depots, needles, capsules, and osmotic pumps. Formulations suitable for parenteral administration include, but are not limited to, aqueous and non-aqueous sterile injection solutions which may contain antioxidants, buffers, bacteriostats and solutes which render the formulation isotonic with the blood of the intended recipient; and aqueous and non-aqueous sterile suspensions that may include suspending agents and thickening agents. Extemporaneous injection solutions and suspensions may be prepared, for example, from sterile powders, granules and tablets.

The invention has been described in detail with particular reference to preferred embodiments thereof, but it will be understood that variations and modifications can be effected within the spirit and scope of the invention. In the drawings and specification, there have been disclosed typical preferred embodiments of the invention. Although specific terms are employed, they are used in a generic and descriptive sense only and not for purposes of limitation, the scope of the invention being set forth in the following claims.

### C. Examples

The following examples are offered for illustrative purposes only.

Hydroxybutenyl-β-cyclodextrin (HBenBCD, MS=4.7) was prepared according to the general methods described in U.S. Pat. No. 6,479,467. Sulfobutyl ether-β-cyclodextrin (SBEBCD, MS=7.0) was prepared according to US 5,376,645. Hydroxypropyl-β-cyclodextrin (HPBCD, MS=4.4) was obtained from Aldrich. All of the cyclodextrin derivatives were dried at 10-15 mm Hg at room temperature for 14 to 60 h prior to use. Drugs were obtained from Apin Chemicals and characterized prior to use. Sporanox® (Janssen Pharmaceutica N.V.) was obtained by prescription. The Sporanox® capsule formulation is a solid formulation filled into hard gelatin capsules (100 mg of itraconazole coated on sugar spheres. Inactive ingredients are gelatin, hydroxypropyl methylcellulose, polyethylene glycol (PEG) 20,000, starch, sucrose, titanium dioxide, FD&C Blue No. 1, FD&C Blue No. 2, D&C Red No. 22 and D&C Red No. 28.). Sporanox® is also offered as an oral solution (10 mg of itraconazole/mL) containing 400 mg/mL of HPBCD. Sporanox® Oral Solution is clear and yellowish in color with a target pH of 2. Other ingredients are hydrochloric acid, propylene glycol, purified water, sodium hydroxide, sodium saccharin, sorbitol, cherry flavor 1, cherry flavor 2 and caramel flavor.

The water used in the following examples was pre-filtered through a Milli-Q Water System (Millipore Corporation) and had very low total organic and pyrogen content and low ionic strength. Buffers were prepared using this same Millipore water. The pH given for the media are approximate values for reference purposes. The actual pH for each solution is provided where appropriate.

Reported pKa values are those obtained from Advanced Chemistry Development Software (ACD). Traditional dissolution profiles were obtained by USP 28-NF 23 711 (United States Pharmacopeia, 2004).

### 1. Example 1

### Determination of the Equilibrium Solubility of Itraconazole in Water and Buffers at 10 wt % Cyclodextrin Concentration

Stock solutions of cyclodextrins were prepared by dissolving an accurately weighed amount of previously dried cyclodextrin in water or in buffer to give a final cyclodextrin concentration of 10 wt %. In this example, the cyclodextrins were HBenBCD, HPBCD, and SBEBCD. The buffers were 0.05 M pH3 phosphate, 0.05 M pH 3 citrate, and 0.05 M pH 4.8 acetate.

The following is a typical procedure for determining the solubility of a drug in a given solvent. To each well of a 2 mL 96-well polypropylene mixing plate was added excess drug (ca. 5-10 mg). The drug was typically added by using a metal loading plate having cavities that will hold an approximate predetermined amount of drug. The cavities on the loading plate were arranged so that they corresponded to the individual wells of the 96-well mixing plate. A line of drug was placed at one end of the loading plate and a glass plate was used to distribute the drug into each cavity. The 96-well mixing plate was placed on the loading plate then inverted and tapped so that the drug dropped into the appropriate well of the mixing plate.

To each well of the pre-loaded 96-well mixing plate was added 300-500 μL of water or buffer (blanks with no CD) or the appropriate CD solution. Typically, each determination was in triplicate. The blanks were used to determine the intrinsic solubility (Sₒ) of drug in that solution and the well containing the CD solutions were used to determine the solubility of drug due to CD (S_{w}). After addition of the stock solutions, the plate was sealed using aluminum foil with a non-volatile adhesive on one surface. The plate was placed on a rotary shaking plate (Helidolph Titramax 1000) and the plate was shaken at 800-1200 rpm at 23° C.±2° for 48-72 h. During the mixing period, the plate was inspected to insure that each well contained undissolved, excess drug. Additional drug was added if necessary.

Following the mixing period, the solutions in each well were transferred to the corresponding wells of a 96-well 2 mL multiscreen filter plate using a multi-channel pipette. The bottom of each well was a hydrophilic membrane (Millipore Corporation). The filter plate was placed on top of a vacuum manifold and the solutions were filtered at ca. 20 mm Hg into the corresponding wells of a 2 mL storage plate. The duration of the filtration period was typically no more than 60 seconds. The storage plate was then sealed with a silicon mat and samples were removed for analysis as appropriate.

The drug content in each well can be determined by a number of methods. Ultraviolet spectroscopy (UV) or high pressure liquid chromatography (HPLC) is preferred. In the present example, UV spectroscopy was used. Typically, 10-20 μL of drug solution was transferred to the corresponding well of a 96-well measurement plate (UV-STAR plates from Greiner with a spectral range of 190-400 nm) and diluted with 1/1 water/ethanol so that the absorbance was in the linear response range. Measurements were made using a SpectraMax Plus 384 Molecular Devices multi-well plate reader. Absorbance was converted to drug concentration using the appropriate absorbtivity for the drug. The drug concentrations were then exported to the appropriate software package for final analysis. Additionally, the final pH of each well was measured to insure that the pH had not drifted significantly due to lack of buffering capacity.

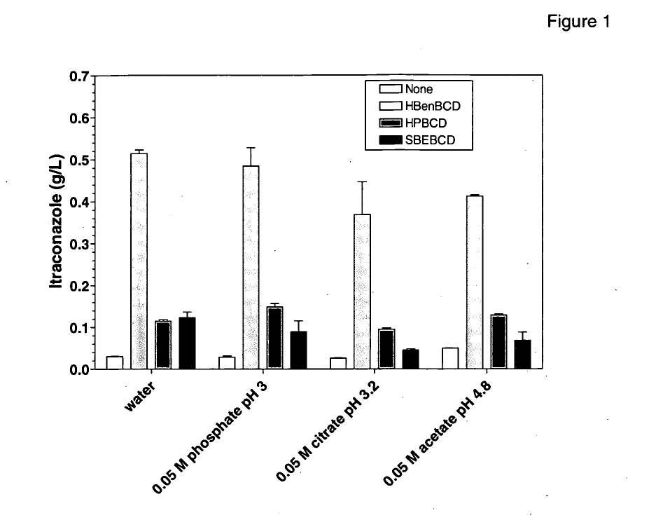

FIG. 1 shows the results for the present experiment involving itraconazole. Clearly, significant differences exist between the cyclodextrins in their ability to solubilize itraconazole. In the case of water, HBenBCD increased the solubility of itraconazole from 30 pg/mL (Sₒ) to 515 pg/mL (S_{w}/Sₒ=17.2). For HPBCD and SBEBCD, the observed S_{w}/Sₒ were 3.8 and 4.2, respectively. Similar differences between HBenBCD and HPBCD, SBEBCD were observed with the buffer solutions. None of the buffers offered significant increases in itraconazole solubility relative to water. The current formulation of itraconazole used clinically involves itraconazole/HPBCD complexes in water (Sporanox®). As this example shows, HBenBCD provides a 4.5X increase in itraconazole solubility relative to HPBCD under identical conditions.

### 2. Example 2

### Determination of Equilibrium Solubility of Itraconazole in Water and pH 3 Phosphate Buffer from 0 to 25 wt % Cyclodextrin Concentrations

The general protocol described in Example 1 was utilized to determine the equilibrium solubility of itraconazole in water and pH 3 phosphate buffers. In this example the concentration of CD was varied from 0 to 25 wt %. The cyclodextrins were HBenBCD, HPBCD, and SBEBCD.

Itraconazole is a racemic mixture of diastereomers with a reported pKa of 6.39. With this pKa, itraconazole would be expected to exhibit higher solubility in acidic media below the pKa. Itraconazole is more difficult to solubilize than many azole antifungal compounds with a similar pKa due to its high crystallinity and melting point (170° C).

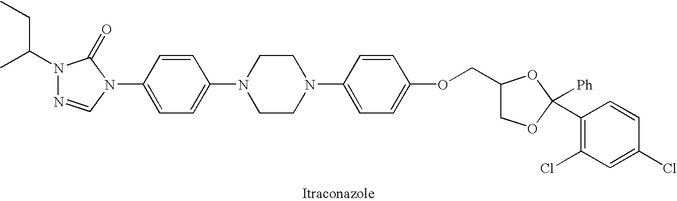

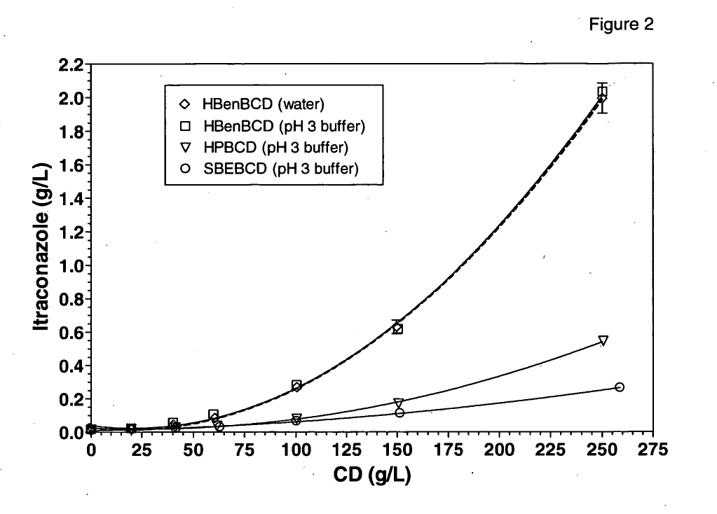

FIG. 2 shows the equilibrium solubility of itraconazole versus cyclodextrin at variable CD concentration. Consistent with results of Example 1, a significant difference was not observed between water and pH 3 phosphate buffer when using HBenBCD as the complexing agent. In this example, HBenBCD provides a significant increase in itraconazole solubility relative to HPBCD or SBEBCD. At ca. 25 wt % CD, the S_{w}/Sₒ ratio for HBenBCD was 132 which was 4X and 5;5X greater than that observed for HPBCD and SBEBCD, respectively. This example demonstrates the differences between cyclodextrin derivatives in their ability to solubilize guest molecules under otherwise identical conditions. The increase in itraconazole solubility using HBenBCD versus HPBCD was significant.

### 3. Example 3

### Determination of the Equilibrium Solubility of Ketoconazole in Water and Buffers at 10 wt % Hydroxybutenyl-β-cyclodextrin Concentration (HBenBCD)

The general protocol described in Example 1 was utilized to determine the equilibrium solubility of ketoconazole in water and buffers at 10 wt % HBenBCD concentration.

Ketoconazole is a racemic mixture of diastereomers with a reported pKa of 6.5. With this pKa, ketoconazole would be expected to exhibit higher solubility in acidic media below the pKa.

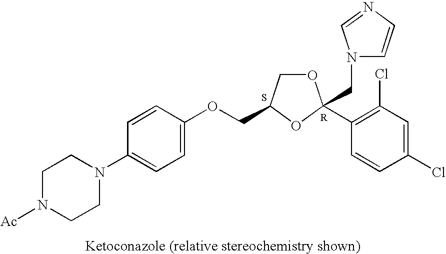

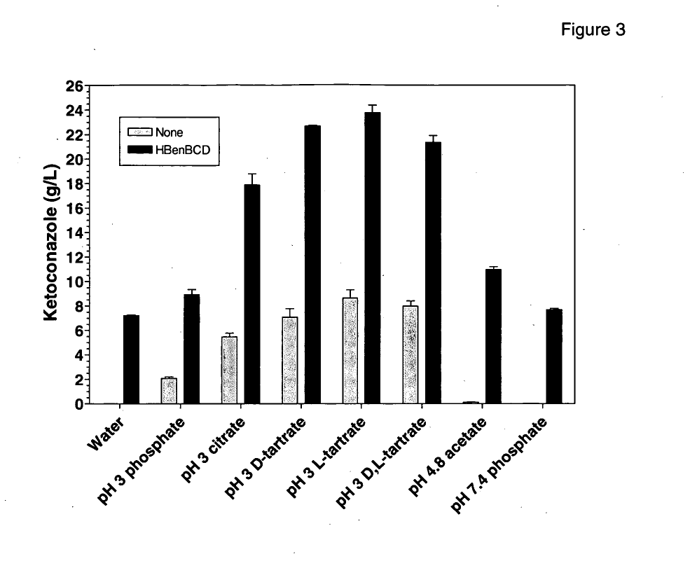

FIG. 3 shows the results obtained with ketoconazole and 10 wt % HBenBCD. The total amount of ketoconazole solubilized with 10% HBenBCD varies with the pH of the media as well as the specific buffer utilized at a given pH. The lowest solubility (S_{w}) was obtained with water and with pH 7.3 phosphate buffer. This is to be expected as the final pH for each of these solutions was greater than 6.5 (Table I). It is interesting to note that S_{w}/Sₒ ratios for these media were significantly greater than the other media. This is due to the low intrinsic solubility of ketoconazole in the media and the ability of HBenBCD to solubilize the drug.

Within the pH 3 media, the lowest ketoconazole solubility (both S_{w} and Sₒ) was found with the phosphate buffer in part due to the lower buffer capacity of the medium (Table I). The highest ketoconazole solubility was observed with the L-tartrate buffer. It should be noted that in general, S_{w}/Sₒ was the lowest for the pH 3 buffers that gave the highest solubilization (S_{w}). This is due to the fact that the intrinsic solubility of the drug in these media was large, which serves to reduce the driving force for complexation by HBenBCD (lower binding constant). Among the tartrate buffers, L-tartrate provided the highest Sₒ and S_{w}. This is not a pH effect as the final pH of the solutions is virtually identical within the Sₒ series and S_{w} series. This would indicate that the chirality of the tartrate impacts its association with the drug and with the drug/HBenBCD complex and that the observed differences are not due to differences in pH.

Finally, with the exception of the pH 3 phosphate buffer, ketoconazole solubility (cf. S_{w} /Sₒ) in pH 4.8 acetate buffer was intermediate to that observed for the pH 3 and pH 7.3 buffers. This is to be expected given the pKa of the drug.

TABLE I
Initial pH, final pH, and S_{w}/Sₒ for each media
used in solubilization of ketoconazole.

| | Initial | Final | Initial | Final | |
|---|---|---|---|---|---|
| | pH No | pH No | pH | pH | S_{w}/ |
| Media^{(a)} | HBenBCD | HBenBCD | HBenBCD | HBenBCD | Sₒ |
| Water | 5.50^{(b)} | 6.56 | 5.50^{(b)} | 7.92 | 600.4 |
| pH 3 phosphate | 2.94 | 3.83 | 3.05 | 5.82 | 4.3 |
| pH 3 citrate | 3.19 | 3.61 | 3.37 | 4.41 | 3.3 |
| pH 3 D-tartrate | 2.97 | 3.49 | 3.07 | 4.17 | 3.2 |
| pH 3 L-tartrate | 2.90 | 3.51 | 3.02 | 4.18 | 2.8 |
| pH 3 D,L-tartrate | 2.90 | 3.46 | 3.02 | 4.14 | 2.7 |
| pH 4.8 acetate | 4.81 | 4.91 | 4.91 | 5.51 | 95.5 |
| pH 7.3 phosphate | 7.27 | 7.31 | 7.30 | 7.34 | 548.9 |

The pH given for the media are for reference purposes. The actual pH for each solution is given in the table. All values are the average of 3 independent measurements.

Due to the low ionic strength of the water, precise measurement of the pH is not possible. The value given is an approximate value that was taken from multiple measurements over an extended period.

### 4. Example 4

### Determination of the Equilibrium Solubility of Voriconazole in Water and Buffers at 10 wt % Hydroxybutenyl-β-cyclodextrin Concentration (HBenBCD)

The general protocol described in Example 1 was utilized to determine the equilibrium solubility of voriconazole in water and buffers at 10 wt % HBenBCD concentration.

Voriconazole is a single diastereomer with two reported pKa values of 4.98 and 12.0. In addition to having low aqueous solubility, voriconazole suffers from stability problems due to retro-aldol reactions in solution.

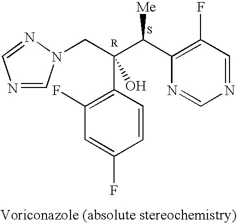

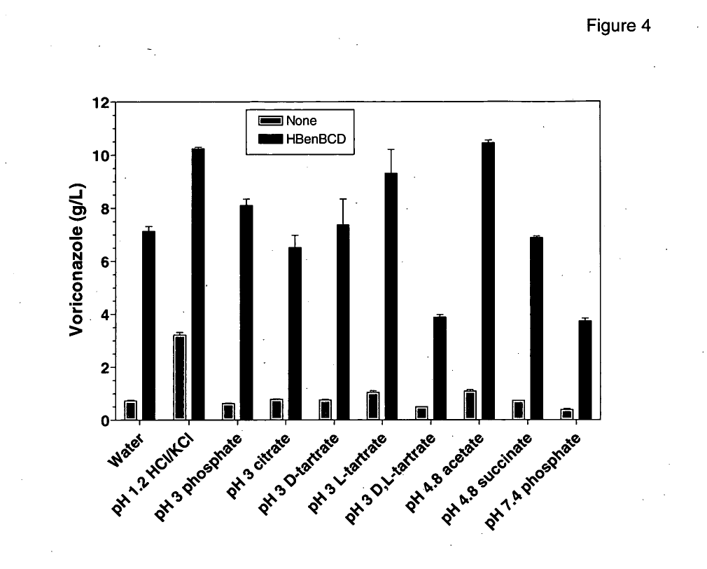

FIG. 4 and Table II shows the results obtained with voriconazole and 10 wt % HBenBCD. As would be expected for a drug with a pKa of 4.98, voriconazole exhibited the highest solubility in pH 1.2 buffer (Sₒ=3.2 mg/mL, S_{w}=10.2 mg/mL). What is surprising is the relatively large increase in voriconazole solubility in the other media over a broad pH range, Regardless of the medium, S_{w}/Sₒ ranged from 7.8 (pH 3 D,L-tartrate) to 13.0 (pH 3 phosphate) indicating that HBenBCD is very effective in solubilizing voriconazole over a wide pH range in many different types of media.

Comparing S_{w} for pH 3 phosphate (inorganic salt), pH 3 L-tartrate (hydroxy dicarboxylic acid), pH 4.8 acetate (carboxylic acid), and pH 4.8 succinate (dicarboxylic acid), the structure of the buffer had no or minor effects on the ability of HBenBCD to solubilize voriconazole. However, within the tartrate family, pH 3 L-tartrate was much more effective (S_{w}=9.3 mg/mL) than either pH 3 D-tartrate (S_{w}=7.4 mg/mL) or pH 3 D,L-tartrate (S_{w}=3.9 mg/mL) in increasing the solubility of voriconazole in the presence of or absence of HBenBCD. As with ketoconazole (Example 3), this observation indicates that the chirality of the tartrate buffers impacts their associations with voriconazole and with the voriconazole/HBenBCD complex. In the case of voriconazole, the chirality effect is more pronounced. These observations can be rationalized if the tartrate is associating with the drug and this species is the actual guest molecule in the drug:HBenBCD complex. In the case of ketoconazole (racemic mixture) the association is weaker than is the case with voriconazole (single diastereomer). That is, matched stereochemical configuration of the drug, buffer, and CD results in a stronger complex versus the case where the stereochemical configuration is mismatched.

In terms of formulations that provide the greatest voriconazole solubility useful over a broad range of physiological conditions, pH 4.8 acetate would be an excellent choice.

TABLE II
Initial pH, final pH, and S_{w}/Sₒ for each media
used in solubilization of voriconazole.

| | Initial | Final | Initial | Final | |
|---|---|---|---|---|---|
| | pH No | pH No | pH | pH | S_{w}/ |
| Media^{(a)} | HBenBCD | HBenBCD | HBenBCD | HBenBCD | Sₒ |
| Water | 5.5^{(b)} | 7.0 | 5.5^{(b)} | 7.6 | 9.8 |
| pH 1.2 HCl/KCl | 1.3 | 1.4 | 1.4 | 1.4 | 3.2 |
| pH 3 phosphate | 3.0 | 3.1 | 3.1 | 3.2 | 13.0 |
| pH 3 citrate | 3.3 | 3.3 | 3.4 | 3.4 | 8.4 |
| pH 3 D-tartrate | 3.0 | 3.1 | 3.1 | 3.2 | 9.8 |
| pH 3 L-tartrate | 3.0 | 3.0 | 3.1 | 3.3 | 8.9 |
| pH 3 D,L-tartrate | 3.0 | 3.1 | 3.1 | 3.1 | 7.8 |
| pH 4.8 acetate | 4.9 | 4.9 | 5.0 | 5.0 | 9.6 |
| pH 4.8 succinate | 4.5 | 5.5 | 4.7 | 5.6 | 9.4 |
| pH 7.3 phosphate | 7.3 | 7.4 | 7.4 | 7.4 | 9.6 |

The pH given for the media are for reference purposes. The actual pH for each solution is given in the table. All values are the average of 3 independent measurements.

Due to the low ionic strength of the water, precise measurement of the pH is not possible. The value given is an approximate value that was taken from multiple measurements over an extended period.

### 5. Example 5

### Determination of the Equilibrium Solubility of Voriconazole in pH 3 Succinate Buffer from 0 to ca. 40 wt % Cyclodextrin Concentrations

The general protocol described in Example 1 was utilized to determine the equilibrium solubility of voriconazole in pH 3 succinate buffer. In this example the concentration of CD was varied from 0 to 40 wt %. The cyclodextrins were HBenBCD and SBEBCD.

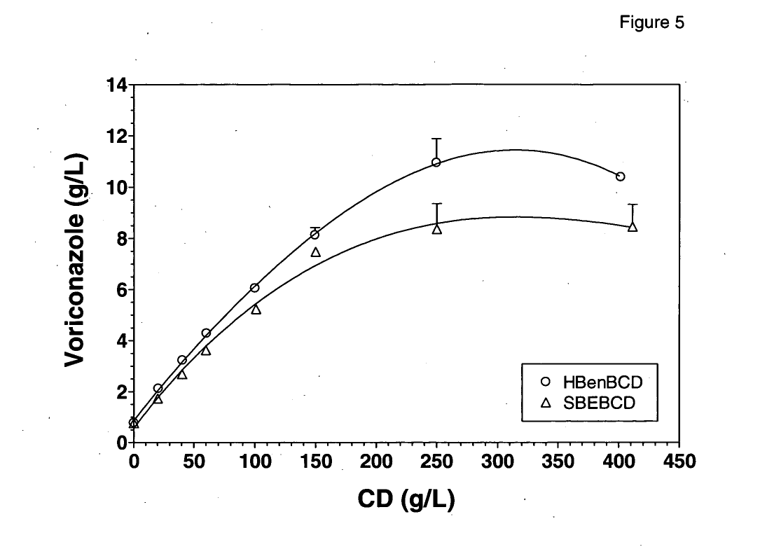

FIG. 5 shows the equilibrium solubility of voriconazole versus cyclodextrin at variable CD concentration. Initially, for both cyclodextrins, the slopes of the curves are linear and there is essentially no difference between the 2 cyclodextrins in their ability to solubilize voriconazole. Beginning at about a SBEBCD concentration of 100 g/L, the SBEBCD curve begins to flatten and ultimately curve downward. HBenBCD behaves similarly except that the curverture does not begin until near 250 g/L of HBenBCD. In both cases, the limitation in voriconazole concentration is due to a limit in complex solubility. Under the conditions of this experiment, at 250 g/L of CD the concentration of voriconazole achieved with HBenBCD was 11 g/L versus 8.6 g/L with SBEBCD. That is, HBenBCD solubilized 25% more voriconazole than SBEBCD. No differences in final pH were observed between the 2 CD/voriconazole series.

This example demonstrates the unexpected superiority of HBenBCD to solubilize antifungal azoles under otherwise identical conditions.

### 6. Example 6

### Determination of the Equilibrium Solubility of Clotrimazole in Water from 0 to ca. 25 wt % Cyclodextrin Concentrations

The general protocol described in Example 1 was utilized to determine the equilibrium solubility of clotrimazole in water. In this example the concentration of CD was varied from 0 to 25 wt %. The cyclodextrins were HBenBCD and HPBCD.

Clotrimazole has a reported pKa of 6.12 with low aqueous solubility from pH 1 to 10. Clotrimazole does not contain a stereogenic center and the chirality of the buffer is not expected to have an effect on drug solubility. The melting point of clotrimazole is 148° C.

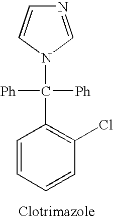

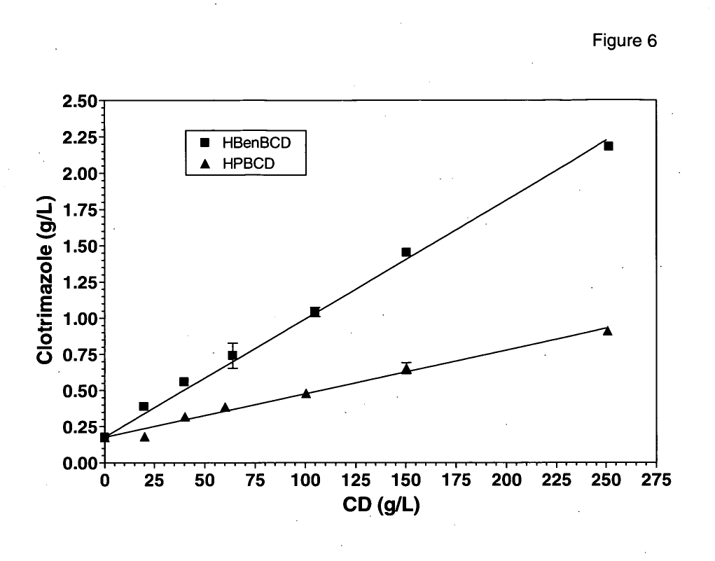

FIG. 6 shows the equilibrium solubility of clotrimazole versus cyclodextrin at variable CD concentration. In this example, HBenBCD provides a significant increase in clotrimazole solubility relative to HPBCD. At ca. 25 wt % CD, the S_{w}/Sₒ ratio for HBenBCD was 12.3 which was 2.4X greater than that observed for HPBCD. This example provides another example of the unexpected superiority of HBenBCD to solubilize antifungal azoles under otherwise identical conditions.

### 7. Example 7

### Preparation and Isolation of Itraconazole:HBenBCD Complexes

The exact methods useful for the preparation of drug:cyclodextrin complexes are highly dependent upon the drug, cyclodextrin, and how the formulation may be used in a clinical setting. The following are examples of procedures used to prepared azole antifungal:HBenBCD complexes for preclinical use using itraconazole as an example. These complexes were utilized in subsequent examples of the present invention.

Itraconazole:HBenBCD complex for IV administration

A 58.8 wt % aqueous HBenBCD solution was prepared by dissolving 10 g of previously dried HBenBCD in 7 g of water.

A second solution was prepared by first adding 900 μL of concentrated HCl to 10 mL of propylene glycol (dried over 4 Å molecular sieves). To a glass vial containing 250.6 mg of itraconazole was added 1000 μL of the propylene glycol/HCl solution. The mixture was vortexed until a homogeneous solution was obtained.

The itraconazole/propylene glycol solution was then slowly added to the HBenBCD aqueous solution with vigorous mixing giving a homogeneous solution. The pH of the solution was then adjusted to pH 4.5 by the slow addition of 0.1 M NaOH with vigorous mixing. The resulting solution was then filtered through a 0.45 μm filter into 25 mL volumetric flask and the solution was diluted to 25 mL. The final formulation contained 40 wt % HBenBCD, 3.6 wt % propylene glycol, and 0.78 wt % itraconazole at pH 4.5.

Itraconazole: HBenBCD solid complex prepared in water/EtOH

A 55.6 wt % aqueous HBenBCD solution was prepared by dissolving 5 g of previously dried HBenBCD in 4 g of water.

A second solution was prepared by first adding 900 μL of concentrated HCl to 10 mL of absolute ethanol. To a glass vial containing 700 mg of itraconazole was added 2000 μL of the ethanol/HCl solution. The mixture was vortexed until a homogeneous purple solution was obtained.

The itraconazole/EtOH solution was then slowly added to the HBenBCD aqueous solution with vigorous mixing giving a homogeneous pale tan solution. The pH of the solution was then adjusted to pH 2.0 by the slow addition of 0.1 M NaOH with vigorous mixing. The resulting solution was then filtered through a 0.45 μm filter into a freeze dry flask. Freeze drying at ca. −45° C., 130 mtorr gave 5.45 g of a white powder containing 12.6 wt % itraconazole.

Itraconazole:HBenBCD solid complex prepared in water

A 40 wt % HBenBCD solution was prepared by dissolving 10 g of HBenBCD in 25 mL of water. To this solution was added 224 mg of itraconazole. The mixture was vortexed then placed in an ultrasonic bath for 30 min at 50° C. for 30 min. The mixture was then placed on a rotary shaker at 27° C. (230 rpm) for ca. 18 h. During this time, the pH of the water rose to 9.5. The pH was adjusted to 5.67 with 0.2 N HCl and the sample was returned to the shaker. After mixing for 24 h the pH had risen to 6.0. The pH was adjusted to 4.3 and the sample was returned to the shaker. After 24 h of mixing, the pH was 4.5. Excess itraconazole was removed by filtration of t he mixture through a 0.45 μm filter into a freeze dry flask. After freeze drying, the resulting white powder contained 1.15 wt % itraconazole.

Itraconazole:HBenBCD PEG400 solution

A 41 wt % HBenBCD PEG400 solution was prepared by dissolving 5 g of previously dried HBenBCD in 7.2 g of PEG400 (dried over 4 Å molecular sieves).

A second solution was prepared by first adding 900 μL of concentrated HCl to 10 mL of propylene glycol (dried over 4 Å molecular sieves). To a glass vial containing 701 mg of itraconazole was added 2400 μL of the propylene glycol/HCl solution. The mixture was vortexed until a homogeneous solution was obtained.

The itraconazole/propylene glycol solution was then slowly added to the HBenBCD PEG400 solution with vigorous mixing giving a homogeneous solution. The pH of the solution was then adjusted to pH 2.5 by the slow addition of propylene glycol containing 9.2 wt % KOH with vigorous mixing. The resulting solution was completely clear and did not require filtration. The final formulation contained 25 wt % HBenBCD, 38 wt % propylene glycol, and 3.75 wt % itraconazole in PEG400 at pH 2.5.

### 8. Example 8

### Determination of the Dissolution Profiles of Itraconazole: HBenBCD Complexes

A solid inclusion itraconazole:HBenBCD complex (10.5 wt % itraconazole) was prepared by a modified method of Example 7. The method was modified by including chitosan (5 wt %) in the HBenBCD solution.

A solid inclusion itraconazole:HBenBCD complex (12.7 wt % itraconazole) prepared by the method of Example 7, a Sporanox® solid formulation, and the itraconazole:HBenBCD complex containing 5 wt % chitosan were filled into hard shell Torpac Lock Ring Gel Capsules (Size #0). Dissolution testing was done according to USP 38-NF 23 711 (United States Pharmacopeia, 2004) using an apparatus with Teflon coated paddles and 500 ml of a USP pH 1.2 buffer solution. The buffer solution was heated to 42° C., followed by vacuum filtration through a 0.45 micron nylon membrane and the vacuum held for an addition 5 minutes. Buffer solution (500 mL) was added to each of the 1000 mL glass dissolution vessels, covered and allowed to equilibrate to 37° C. for 30 minutes. The vessels were kept at constant temperature by a water bath kept at 37° C. The capsules were weighted down with a Varian 3-prong capsule weight and added to the dissolution vessels. Once the capsules sunk to the bottom of the vessels, the test was initiated by turning the paddles at 100 rpm. The testing was done by withdrawing samples as a function of time. The removed samples were filtered through a 0.45 micron membrane filter and placed in a 96-well measurement plate (UV-STAR plates from Greiner with a spectral range of 190-400 nm). Measurements were made using a SpectraMax Plus 384 Molecular Devices multi-well plate reader. The concentrations measured were then used to calculate the percentage of drug released.

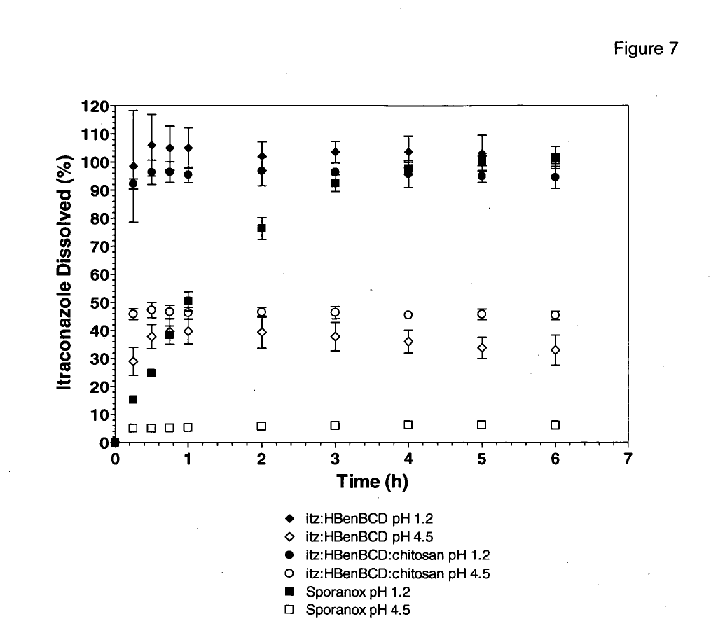

FIG. 7 shows the dissolution profiles obtained for Sporanox® solid formulation, the itraconazole:HBenBCD complex, and the itraconazole:HBenBCD complex containing 5 wt % chitosan. In the case of the Sporanox® solid formulation, at pH 1.2 itraconazole slowly dissolved reaching 100% dissolution at ca. 4 h. In contrast, both of the itraconazole:HBenBCD complexes reached 100% dissolution in ca. 30 min. No itraconazole precipitation was observed over the course of the 6 h experiment. At pH 4.5, only ca. 6% of the itraconazole dissolved with the Sporanox® solid formulation. In the case of the itraconazole:HBenBCD formulation, dissolution was rapid at pH 4.5 and a concentration of 33% was achieved at 6 h. With the itraconazole:HBenBCD formulation containing 5 wt % chitosan, dissolution was again rapid at pH 4.5 but in this case, a concentration of 45% was achieved at 6 h.

As indicated by FIG. 7, at pH 1.2 (stomach pH), both of the itraconazole:HBenBCD complexes dissolved much more rapidly than did the Sporanox® solid formulation. At pH 4.5 (pH of the upper GI tract) with the itraconazole:HBenBCD complexes, much more of the itraconazole rapidly dissolved relative to the Sporanox® solid formulation. In the case of the itraconazole complex formulated with 5 wt % chitosan, a significantly higher concentration of itraconazole was achieved relative to the itraconazole:HBenBCD complex that did not contain chitosan.

This example illustrates that with the itraconazole:HBenBCD complexes, a higher concentration of itraconazole can be achieved more rapidly over a wider pH range than can be with the Sporanox® solid formulation. In the case of the itraconazole:HBenBCD complex formulated with chitosan, an even higher concentration of itraconazole was achieved at pH 4.5 relative to when chitosan is absent. More rapid dissolution of itraconazole over a wider pH range is important as this can serve as a means to increase the drug absorption window and increase oral bioavailability.

### 9. Example 9

### Pharmacokinetic Study Involving Administration of Itraconazole:HBenBCD Complexes to Sprague-Dawley Rats

A pharmacokinetic study involving administration of itraconazole/HBenBCD complexes to male Sprague-Dawley rats was completed. The complexes were prepared by the methods of Example 7. The study consisted of three groups, divided by the route of administration (oral capsule, oral solution, or intravenous), of 3 male rats (n=3). Each rat received a single 20 mg/kg of itraconazole. All of the animals were fasted prior to dose administration by removing food from their cages 14 hours before dose administration. Food was presented again 3 hours after dose administration. During this fasting period, water was removed from the rat cages 30 minutes prior to dose administration and returned 1 hour after dose administration. All groups received 0.5 mL of pH 1.8 water (pH adjusted with HCl) by oral gavage 30 minutes prior to dosing. The liquid doses were given via 1 mL syringes possessing a measurement capability of ±0.01 mL at a dose volume of 2 Ml/kg body weight. For the IV group (0.78 wt % itraconazole), the infusion rate was ca. 0.3 mL per minute via the tail vein. For the oral solution group (0.81 wt %), the dose was given as a 1 minute bolus. In the case of the oral capsule group, size 9 mini capsules (Torpac, Fairfield, N.J.) were filled with itraconazole/HBenBCD (126 mg/g) complex. The dose was administered by placing the capsule in the holding cup of a dosing syringe. The tip of the dosing syringe was then inserted into the rat esophagus and the capsules were expelled into the rat stomach with the plunger of the dosing syringe. Each rat from all groups was given 1.0 mL of pH 1.8 water (pH adjusted with HCl) immediately following dosing. Blood samples (ca. 0.22 mL) were taken via the jugular vein at different time points into tubes containing EDTA by automated blood sampling using a DiLab AccuSampler. For the IV group, blood samples were taken at 0.5,1, 2, 3, 4, and 8 h. For the oral groups, blood samples were taken at 1, 2, 3, 4, 6, and 12 h. Plasma was separated and stored in 96 well plates at −17 to −80° C. until analysis. The concentration of itraconazole and metabolites of itraconazole in the plasma were determined using LC-MS-MS spectrometry. Plasma (100 μL) was precipitated with 250 μL of acetonitrile. After centrifugation, the supernatant was analyzed for itraconazole and hydroxyitraconazole by reversed phase LC-MS-MS using ketoconazole as an internal standard.

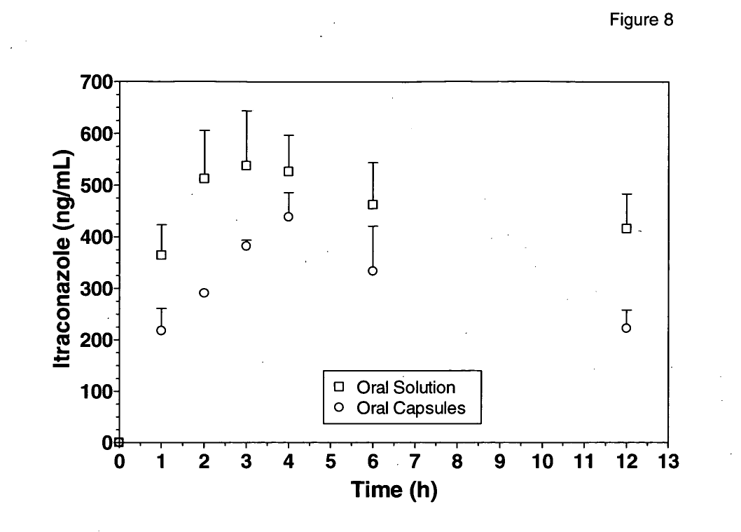

FIG. 8 shows the concentration of itraconazole in plasma versus time for the groups receiving oral doses of itraconazole:HBenBCD complex. The pharmacokinetic data for this experiment is also summarized in Table III. As can be seen, the time (Tₘₐₓ) to reach maximum itraconazole plasma concentration (Cₘₐₓ) was 3 h for the group receiving the oral solution and 4 h for the group receiving the oral capsules. Using the area under the plasma concentration-time curve (AUC) for the IV group and the oral groups, the oral bioavailability of itraconazole was found to be ca. 52% (oral solution) and 34% (oral capsules). The oral bioavailability of itraconazole in Sprague-Dawley rats in similar itraconazole:HPBCD formulations has been studied by Lee (Antimicrobial Agents and Chemotherapy, 2004, 48,1756-1762; Biopharm. Drug Dispos., 2003, 24, 63-70). Lee found the oral bioavailability of itraconazole from oral solution doses to be ca. 35 and 32%, respectively, compared to ca. 52% found in present study. This example illustrates that HBenBCD is very effective in increasing the bioavailability of itraconazole in a variety of different dosage forms. These dosage forms provided unexpectedly increased bioavailability relative to equivalent dosage forms in prior studies.

TABLE III
Pharmacokinetic data for Sprague-Dawley rats
receiving 20 mg/Kg doses of itraconazole.

| | | Cₘₐₓ | | Oral |
|---|---|---|---|---|
| | AUC_{(0 to ∞)} | (ng/ | Tₘₐₓ | Bioavail- |
| Species-Group | (ng*min/mL) | mL) | (h) | ability |
| Itz - HBenBCD IV | 11035 | | | |
| HItz - HBenBCD IV | 2691 (a) | 409 | 4 | |
| Itz - HBenBCD Oral Solution | 5703 | 538 | 3 | 52% |
| HItz - HBenBCD Oral Solution | 2790 (b) | 292 | 3 | |
| Itz - HBenBCD Oral Capsules | 3764 | 439 | 4 | 34% |
| HItz - HBenBCD Oral Capsules | 2974 (b) | 380 | 4 | |

Abbreviations: Itz, itraconazole; Hitz. hydroxyitraconazole.
(a) 0 to 8 h.
(b) 0 to 12 h.

All references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

We claim:

1. A method of increasing the aqueous solubility of an antifungal azole compound comprising forming a complex or mixture of at least one antifungal azole compound with a hydroxybutenyl cyclodextrin.

2. The method of claim 1 wherein the hydroxybutenyl cyclodextrin is hydroxybutenyl-α, β, or γ-cyclodextrin.

3. The method of claim 2 wherein the hydroxybutenyl cyclodextrin is hydroxybutenyl-β-cyclodextrin.

4. The method of claim 1 wherein the hydroxybutenyl cyclodextrin has a molar substitution of about 1 to about 12.

5. The method of claim 1 wherein the hydroxybutenyl cyclodextrin has a molar substitution of about 3 to about 10.

6. The method of claim 1 wherein the hydroxybutenyl cyclodextrin has a molar substitution of about 4 to about 7.

7. The method of claim 6 wherein the hydroxybutenyl cyclodextrin is water soluble.

8. The method of claim 1 wherein the hydroxybutenyl cyclodextrin is sulfonated hydroxybutenyl cyclodextrin.

9. The method of claim 8 wherein the hydroxybutenyl cyclodextrin is sulfonated hydroxybutenyl-α, β, or γ-cyclodextrin.

10. The method of claim 9 wherein the hydroxybutenyl cyclodextrin is sulfonated hydroxybutenyl-β-cyclodextrin.

11. The method of claim 8 wherein the sulfonated hydroxybutenyl cyclodextrin has a molar substitution of hydroxybutyl sulfonate of about 0.02 to about 12.

12. The method of claim 8 wherein the sulfonated hydroxybutenyl cyclodextrin has a molar substitution of hydroxybutyl sulfonate of about 0.1 to about 2.

13. The method of claim 1 wherein the antifungal azole compound is an imidazole or triazole compound.

14. The method of claim 13 wherein the antifungal azole compound is albaconazole, becliconazole, bifonazole, bilastine, butoconazole, carbendazim, clotrimazole, doconazole, eberconazole, econazole, fenticonazole, fluconazole, flutrimazole, fosfluconazole, genaconazole, imiquimod, itraconazole, ketoconazole, lanoconazole, liarozole, luliconazole, metronidazole, miconazole, mycoprex, omoconazole, parconazole, posaconazole, ravunconazole, resiquimod, rifaximin, samidazole, saperconazole, seraconazole, terconazole or voriconazole or structural analogs or metabolites thereof.

15. The method of claim 14 wherein the antifungal azole compound is itraconazole.

16. The method of claim 14 wherein the antifungal azole compound is voriconazole.

17. The method of claim 14 wherein the antifungal azole compound is ravunconazole.

18. The method of claim 14 wherein the antifungal azole compound is posaconazole.

19. The method claim 1 wherein the solubility of the complex or mixture of at least one antifungal azole compound with a hydroxybutenyl cyclodextrin is from about 1.1 to about 1000 times greater than the aqueous solubility of the antifungal azole compound without the hydroxybutenyl cyclodextrin.

20. The method of claim 19 wherein the solubility of the complex or mixture of at least one antifungal azole compound with a hydroxybutenyl cyclodextrin is from about 2 to about 100 times greater than the aqueous solubility of the antifungal azole compound without the hydroxybutenyl cyclodextrin.

21. The method of claim 19 wherein the solubility of the complex or mixture of at least one antifungal azole compound with a hydroxybutenyl cyclodextrin is from about 2 to about 50 times greater than the aqueous solubility of the antifungal azole compound without the hydroxybutenyl cyclodextrin.

22. The method of claim 19 wherein the solubility of the complex or mixture of at least one antifungal azole compound with a hydroxybutenyl cyclodextrin is from about 2 to about 25 times greater than the aqueous solubility of the antifungal azole compound without the hydroxybutenyl cyclodextrin.

23. The method of claim 19 wherein the solubility of the complex or mixture of at least one antifungal azole compound with a hydroxybutenyl cyclodextrin is from about 2 to about 5 times greater than the aqueous solubility of the antifungal azole compound without the hydroxybutenyl cyclodextrin.

24. A method of increasing the aqueous solubility of an antifungal azole compound comprising forming a complex or mixture of an antifungal azole compound with a hydroxybutenyl cyclodextrin in the presence of a chiral compound or salt.

25. The method of claim 24 wherein the chiral compound is citric acid or a salt thereof.

26. The method of claim 24 wherein the chiral compound is tartaric acid or a salt thereof.

27. The method of claim 26 wherein the chiral compound is D-tartaric acid or a salt thereof.

28. The method of claim 26 wherein the chiral compound is L-tartaric acid or a salt thereof.

29. The method of claim 24 further comprising matching the stereochemical configuration of the antifungal azole compound, the hydroxybutenyl cyclodextrin and the chiral compound.

30. A method of increasing the bioavailability of an antifungal azole compound comprising administering a complex or mixture of an antifungal azole compound with a hydroxybutenyl cyclodextrin to a subject.

31. The method of claim 30 wherein the hydroxybutenyl cyclodextrin is hydroxybutenyl-α, β, or γ-cyclodextrin.

32. The method of claim 31 wherein the hydroxybutenyl cyclodextrin is hydroxybutenyl-β-cyclodextrin.

33. The method of claim 30 wherein the hydroxybutenyl cyclodextrin has a molar substitution of about 1 to about 12.

34. The method of claim 30 wherein the hydroxybutenyl cyclodextrin has a molar substitution of about 3 to about 10.

35. The method of claim 30 wherein the hydroxybutenyl cyclodextrin has a molar substitution of about 4 to about 7.

36. The method of claim 35 wherein the hydroxybutenyl cyclodextrin is water soluble.

37. The method of claim 30 wherein the hydroxybutenyl cyclodextrin is sulfonated hydroxybutenyl cyclodextrin.

38. The method of claim 37 wherein the hydroxybutenyl cyclodextrin is sulfonated hydroxybutenyl-α, β, or γ-cyclodextrin

39. The method of claim 38 wherein the hydroxybutenyl cyclodextrin is sulfonated hydroxybutenyl-β-cyclodextrin.

40. The method of claim 37 wherein the sulfonated hydroxybutenyl cyclodextrin has a molar substitution of hydroxybutyl sulfonate of about 0.02 to about 12.

41. The method of claim 37 wherein the sulfonated hydroxybutenyl cyclodextrin has a molar substitution of hydroxybutyl sulfonate of about 0.1 to about 2.

42. The method of claim 30 wherein the antifungal azole compound is an imidazole or triazole compound.

43. The method of claim 42 wherein the antifungal azole compound is albaconazole, becliconazole, bifonazole, bilastine, butoconazole, carbendazim, clotrimazole, doconazole, eberconazole, econazole, fenticonazole, fluconazole, flutrimazole, fosfluconazole, genaconazole, imiquimod, itraconazole, ketoconazole, lanoconazole, liarozole, luliconazole, metronidazole, miconazole, mycoprex, omoconazole, parconazole, posaconazole, ravunconazole, resiquimod, rifaximin, samidazole, saperconazole, seraconazole, terconazole or voriconazole or structural analogs or metabolites thereof.

44. The method of claim 43 wherein the antifungal azole compound is itraconazole.

45. The method of claim 43 wherein the antifungal azole compound is voriconazole.

46. The method of claim 43 wherein the antifungal azole compound is ravunconazole.

47. The method of claim 43 wherein the antifungal azole compound is posaconazole.

48. The method of claim 30 wherein the complex or mixture of an antifungal azole compound with a hydroxybutenyl cyclodextrin further comprises an amine-containing polymer.

49. The method of claim 48 wherein the amount of the amine-containing polymer in the complex or mixture is from about 3 wt % to about 40 wt %.

50. The method of claim 49 wherein the amount of the amine-containing polymer in the complex or mixture is from about 5 wt % to about 30 wt %.

51. The method of claim 48 wherein the amine-containing polymer is chitosan or a derivative thereof.

52. The method of claim 30 wherein the subject is a human.

53. A method of increasing the bioavailability of an antifungal azole compound comprising administering a complex or mixture of an antifungal azole compound with a hydroxybutenyl cyclodextrin produced by the method of claim 24 to a subject.

54. The method of claim 53 wherein the subject is a human.

55. A method of increasing the bioavailability of an antifungal azole compound comprising administering a complex or mixture of an antifungal azole compound with a hydroxybutenyl cyclodextrin produced by the method of claim 29 to a subject.

56. The method of claim 55 wherein the subject is a human.
